(19) United States
(12) Patent Application Publication
(10) Pub. No.: US 2010/0312260 A1
(43) Pub. Date: Dec. 9, 2010

(54) INTERRUPTED TISSUE APPOSITION DEVICES

(76) Inventor: Daniel M. Herron, Port Washington, NY (US)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 12/793,027

(22) Filed: Jun. 3, 2010

Related U.S. Application Data
(60) Provisional application No. 61/183,750, filed on Jun. 3, 2009.

(51) Int. Cl. A61B 17/04

(52) U.S. Cl. 606/145; 606/144

(57) ABSTRACT

An interrupted suturing device for applying one or more suture elements to tissue is provided. The device includes a handle and a shaft extending from the handle. The shaft includes a suture channel and a tip member that is coupled to the shaft and movable between an extended position and a retracted position. The tip member has a return channel, where open ends of the suture channel are axially aligned with open ends of the return channel. A drive mechanism is operatively coupled to the actuator to drive the suture element along the suture and return channels, while the tip member is operatively coupled to the drive mechanism to cause movement of the tip member between the extended and retracted positions.

### CROSS REFERENCE TO RELATED APPLICATIONS

This application claims the benefit of U.S. Provisional Application Ser. No. 61/183,750, filed Jun. 3, 2009, the contents of which are incorporated by reference herein.

### BACKGROUND OF THE INVENTION

1. Field of the Invention

The present disclosure relates to surgical instruments for inserting interrupted sutures into human and/or animal tissue, and more particularly, to tissue-apposition devices, interrupted suture elements, and methods for using such devices and elements.

2. Description of Related Art

A common surgical procedure is a tissue apposition procedure, where edges of human and/or animal tissue are placed in close proximity to one another and secured to one another. One method of securing apposed tissue is the use of a suture to hold tissue, such as, but not limited to, skin, internal organs, blood vessels and other tissues of the human and/or animal body, together after the tissue has been severed by injury, incision or surgery.

Suturing is one of the most commonly performed steps in any surgical procedure, and is currently accomplished with standard suture material attached to a steel needle. Suturing of tissues is accomplished by the surgeon passing the sharpened tip of a needle with a suture attached thereto through the tissue segments such that the needle tip penetrates the tissue segments causing the needle and suture material to span the incision or tissue gap requiring closure. The surgeon then pulls the needle through the tissue segments causing a portion of the attached suture to follow the path of the needle.

After the needle and the portion of the suture are passed through the tissue, the suture may be tied to itself and cut, creating a simple interrupted suture (“interrupted suture”). This process may be repeated as needed to close the incision with a plurality of interrupted sutures, each tied separately.

Of course, other stitching techniques other then the interrupted suture are known. For example, the first suture may be tied without cutting and the remaining length of suture material passed through the tissue additional times to create a running suture or stitch. The running suture may then be tied to itself at the trailing end of the incision, completing the closure. Still other suturing techniques are known such as, but not limited to the mattress stitch, the horizontal mattress stitch, the vertical mattress stitch, the FIG. 8 stitch, the continuous locking stitch, the subcuticular stitch, and others.

Further, other non-suturing tissue apposition processes are also known. These non-suturing processes can include processes such as, but not limited to, stapling processes, chemical bonding processes, and arterial closure devices, and others.

Each suturing and non-suturing apposition process (e.g., suturing, stapling, chemical bonding, arterial closure devices, etc) has benefits and drawbacks, which lend each process to particular uses. Nonetheless, interrupted suturing remains a very popular and necessary tissue apposition process.

Even though interrupted suturing processes have been used for many years and are well-accepted as a standard approach, interrupted suturing processes present a number of major disadvantages. For example, interrupted suturing processes suffer from disadvantages including lack of speed; technical difficulty, especially in anatomic locations that are hard to reach or see; difficulty in performing and teaching in laparoscopic surgery; operator-dependancy of knot consistency and quality; possibility of knots slipping inadvertently; difficulty in tightening knots further once they are applied such that a loose knot will typically need to be cut out and replaced; and because of the recently established eighty-hour workweek for residents, decreased time is available for teaching surgical trainees complex surgical skills such as open and laparoscopic knot-tying.

In addition, interrupted suturing processes require the use of standard needles that pass the suture through tissue. The use of exposed, sharp needles can cause injury to the patient's anatomy during suturing or knot-tying; the exposed needle presents a risk of injury to the surgeon, the surgical assistant, and the scrub technician as it is passed to and from the surgical field—needle stick injuries present the risk of transmission of blood borne infections, including potentially fatal hepatitis B and C, HIV, and others; needles may be difficult to pass through laparoscopic ports; and needles are difficult to maneuver using laparoscopic instruments.

Accordingly, there is a need for interrupted tissue apposition devices and interrupted suture elements that overcome, alleviate, and/or mitigate one or more of the aforementioned and other deleterious effects of prior art interrupted suturing processes and devices.

### BRIEF SUMMARY OF THE INVENTION

A tissue apposition device is provided that fastens tissue together and eliminates the need for surgical sutures in operating room situations. The device is, in some embodiments disposable, and includes an applicator that is loaded with a magazine of implantable suture elements. The suture elements can be made of permanent or absorbable surgical material, as required for the given surgical application. The device includes a movable trigger and the suture elements are placed in the tissue with the squeeze of the trigger or the push of a button. Thus, the device advantageously mitigates the need for complex needle placement maneuvers. The device initially places the suture element in a tied but loose condition, where the loose suture element can then be precisely tightened down as needed for the surgical situation. The suture element includes a sharp portion, which the device automatically removes after placement. In addition, the device stores the used and removed sharp portion within a containment area within the device. Thus, the device advantageously obviates the need for handling of sharps and therefore mitigates the risk of needlestick injury.

In some embodiments, the supply suture elements are stored in a removable magazine or cartridge. In this manner, when the supply of suture elements in the device is exhausted, a new magazine may be loaded to allow further suturing. In addition, the use of removable magazines allows the same device to be loaded with suture elements of different properties, as needed, to accommodate different tissue types and surgical requirements.

In one embodiment, a suturing device includes a cartridge having a plurality of elongated suture elements connected together in a head-to-tail manner. Each suture element has a pointed needle portion at a head thereof and a ratchet opening at a tail thereof. The suturing device also includes an applicator that receives the cartridge and includes a drive mechanism for driving the interconnected suture elements along a shaft of the applicator to a tip portion thereof. The tip portion is operatively coupled to the drive mechanism such that the tip portion moves between an open, extended position and a closed, retracted position relative to the shaft. The shaft includes a first channel architecture and the tip portion includes a complementary second channel architecture that allows a lead suture element to pass first through a first tissue segment disposed within a space between the shaft and tip portion before being passed through a second tissue segment disposed within the space after having traveled through the second channel architecture. The first channel architecture includes a channel segment that directs the needle portion of the lead suture element to pass through the ratchet opening formed at the tail portion thereof so as to create a looped suture element that extends across the incised tissue and allow tightening thereof.

In another embodiment, a cartridge for use with a suturing device includes a housing having a hollow interior that contains a plurality of elongated suture elements connected together in a head-to-tail manner. Each suture element has a pointed needle portion at a head thereof and a ratchet opening at a tail thereof. The housing has a slot through which the interconnected suture elements exit.

In yet another embodiment, a suturing device for applying a suture element to tissue includes a handle with an actuator and a shaft extending from the handle. The shaft includes a suture channel that is open at two locations along a distal end of the shaft. The suturing device includes a tip member that is coupled to the shaft and movable between an extended position where the tip member is spaced from the distal end of the shaft and a retracted position. A space is formed between the tip member and the distal end of the shaft in the extended position for receiving a member to be sutured. The tip member has a return channel formed therein. The return channel is open at two locations along a proximal end of the tip member. The open ends of the suture channel are axially aligned with the open ends of the return channel. A drive mechanism is operatively coupled to the actuator for driving the suture element along the suture channel and return channel. In addition, the tip member is operatively coupled to the drive mechanism to cause movement of the tip member between the extended position and retracted position.

These and other aspects, features and advantages shall be apparent from the accompanying drawings and description of various embodiments of the disclosure.

### BRIEF DESCRIPTION OF THE SEVERAL VIEWS OF THE DRAWINGS

The present disclosure is illustrated by way of example and not by way of limitation in the figures of the accompanying drawings in which like references indicate similar elements.

FIG. 1 is a top, side perspective view of an exemplary embodiment of an interrupted tissue apposition device according to the present disclosure, shown in an extended or open position;

FIG. 2 is a top perspective view of a suture element according to one embodiment;

FIG. 3 is bottom perspective view of the suture element of FIG. 2;

FIG. 4 is side elevation view of the suture element of FIG. 2;

FIG. 5 is a top plan view of an interconnected supply of suture elements arranged in a head-to-tail manner; and

FIG. 6 is a top plan view, in cross-section, of a cartridge that stores the interconnected supply suture elements of FIG. 5;

FIG. 7 is a close-up partial top, end perspective view of a tip end of the device of FIG. 1;

FIG. 8 is a side elevation view of the device of FIG. 1 in a retracted or closed position;

FIG. 9 is a partial side elevation view, in partial cutaway, of the device of FIG. 1;

FIG. 10 is a partial top, rear perspective view, in partial cutaway, of the device of FIG. 1; and

FIG. 11 is a top plan view of the tip end of the device of FIG. 1 in the extended or open position;

FIG. 12 is a top plan view, in cross section, of the tip end of the device of FIG. 1 in the extended or open position showing the reception of tissue for the suturing thereof.

### DETAILED DESCRIPTION OF THE INVENTION

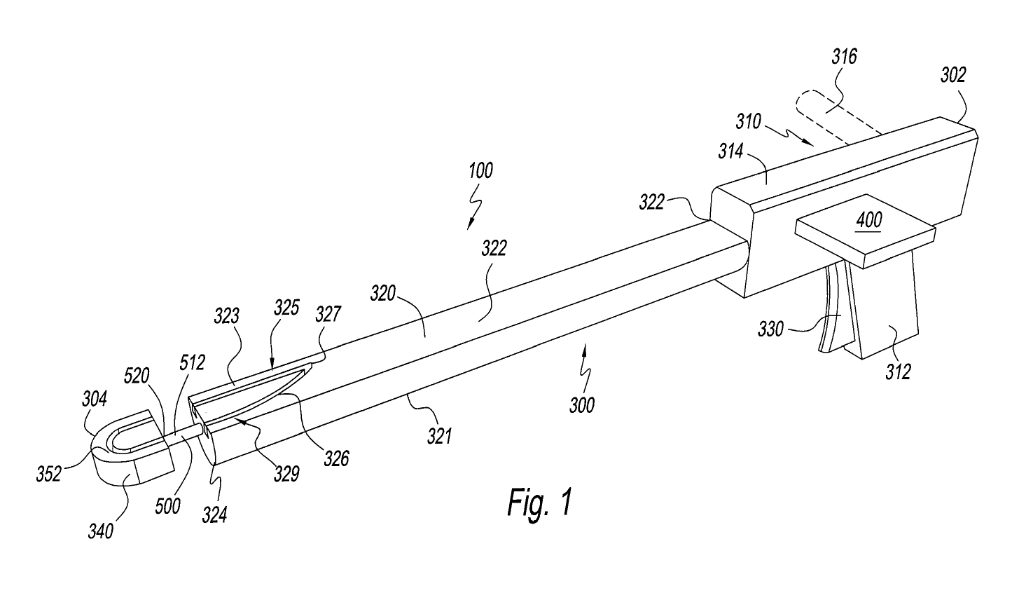

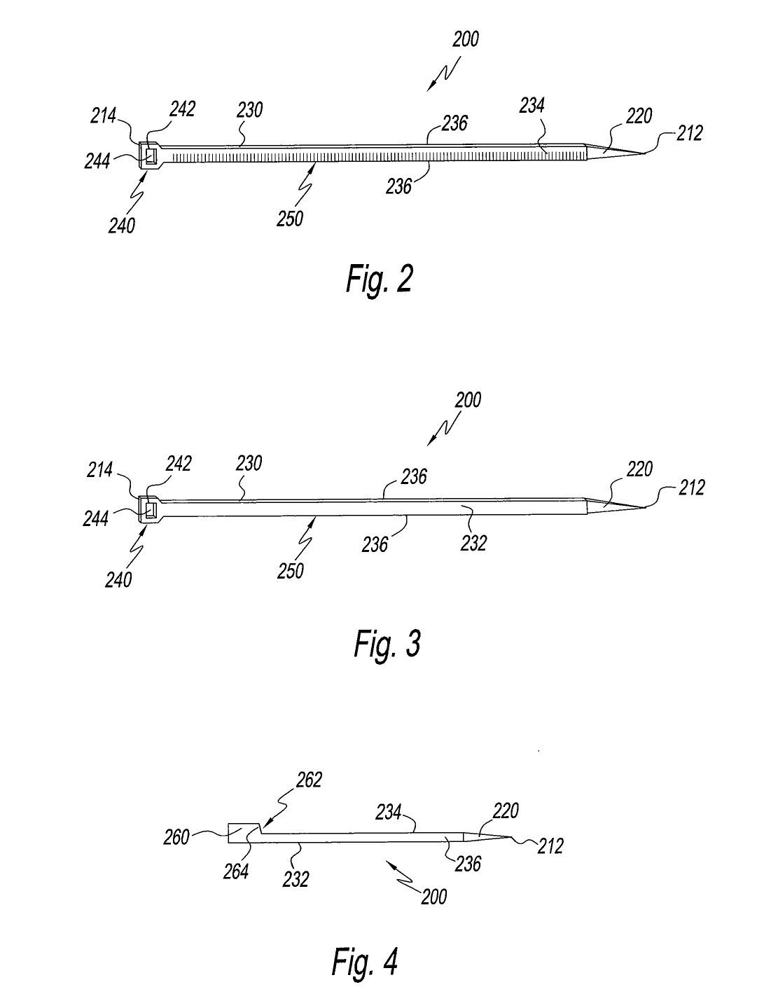

Now turning to FIGS. 1 and 2, an interrupted tissue apposition device according to an exemplary embodiment of the present disclosure is shown and is generally referred to by reference numeral 100. Device 100 can be used to produce an interrupted suture (not shown) by applying one or more suturing elements 200, shown in FIG. 2, into the tissue so as to enable connection tissue segments to one another.

Advantageously, device 100 and suturing elements 200 are superior to existing interrupted suturing technology in many ways: it is faster, easier, simpler, more consistent and reproducible, and safer to both the patient and the surgical team. Device 100 and suturing elements 200 mitigate the need for complex needle placement maneuvers and obviates the need for handling of sharps and therefore mitigates the risk of needlestick injury.

The device 100 is generally formed of two components, namely an applicator 300 and a removable magazine or cartridge 400. Applicator 300 removably receives cartridge 400. Cartridge 400 carries a plurality of suture elements 200 therein, where applicator 300 is configured to selectively apply suture elements 200 to tissue, one at a time, to perform a suturing operation.

It will be appreciated that the elements shown in the present drawings are not shown to scale in order to more clearly convey the teachings of the present disclosure. Device 100 is intended to be used in a number of different surgical operations many of which require the device to be inserted through a small incision. For example, the diameter of the shaft of device 100 can be about 10 to 12 millimeters (mm) to allow it to be inserted into a laparoscopic port having a similar size. Of course, it is contemplated by the present disclosure for device 100 to have any desired size.

Since suture placement and fixation is often the most technically demanding portion of a surgical operation, facilitation of the process using device 100 according to the present disclosure substantially decreases the complexity of a given surgical procedure and the time required to complete it. This improvement is particularly marked in situations that make conventional knot-tying difficult, such as laparoscopic surgery, where all suture manipulation is done intracorporeally using instruments, and hand manipulation of the needle and suture material is not possible. In addition, the elimination of an exposed needle will substantially reduce the risk of injury to both the surgeon and the patient.

Device 100 provides a number of advantages compared to conventional technology, especially suture technology. In particular, device 100 simplifies a very common surgical task; it decreases the skills required to appose tissue; it reduces the time required to tie knots; it provides a more predictable and reproducible closure than tying; and it creates a knot that can be tightened further after it has been applied. Since device 100 eliminates the use of an exposed needle, it reduces the risk of injury to the patient; reduces the risk of injury to the surgeon and other members of the operating room team; and increases compliance with hospital safety directives.

The construction of the exemplary applicator 300 is discussed below after a discussion concerning the construction and packaging of suture element 200 within cartridge 400.

FIGS. 2 through 4 show a first exemplary embodiment of suture element 200 that is intended for use with device 100 and is for application to a surgical site for closure of the segments of incised tissue, etc.

Suture element 200 is in the form of a knotless tissue apposition member that consists of an elongated structure that has a first end 212 and an opposing second end 214. First end 212 can be thought of as a head, while second end 214 can be thought of as a tail. Suture element 200 is formed of different sections or segments that provide different functionality. More specifically, suture element 200 includes a needle 220 at first end 212; a body 230 joining the first and second ends 212, 214; and a coupling section or tail 240 at the second end.

Needle 220 of suture element 200 is in the form a sharp tip of first end 212 of the suture element, which is sufficient to penetrate tissue. Needle 220 can be manufactured from any number of different types of material that are suitable for the intended use, namely for inserting interrupted sutures. Needle 220 is flexible in that it is designed to readily flex about a horizontal plane in FIG. 2; however, the needle 220 does not readily flex about a vertical plane in FIG. 2. Needle 220 is sharp at its pointed tip located at first end 212. However, it is contemplated by the present disclosure for needle 220 to be blunt or sharp for use in dense or soft tissue respectively, as required. Preferably, needle 220 has substantially the same dimensions where it interfaces with body 230 to facilitate penetration of the tissue being sutured.

In one embodiment, needle 220 can be made of a flexible material that is removably connected to body 230 of suture element 200. Alternatively, needle 220 can be made of the same material as body 230 of suture element 200, but can be removed therefrom. For example, needle 220, as well as body 230 and tail 240, can be formed of polypropylene, polyglycolic acid or other permanent or temporary material that is suitable for use in the intended application. Needle 220 thus represents a tip that is sharp enough to facilitate passage through tissue.

As described herein below, since needle 220 is separated from body 230 by device 100 after insertion of suture element 200 into the incised tissue, the needle 220 it can be formed of any permanent material as needed. Needle 220 can therefore be made of metal or any other material so long as it is flexible enough to pass through a curved feed track of device 100. In some embodiments, needle 220 can be formed of a material that is radio-opaque such that it can be identified on an X-ray or other imaging device, as needed.

In addition, needle 220 has sufficient width in the lateral dimensions to create strength, but narrow in the vertical dimension to facilitate flexing as suture element 200 is driven through device 100. As described in more detail below, needle 220 is sized and configured so that the needle can be advanced through the guide channel of device 100.

Body 230 is the longest segment of suture element 200 and is formed between needle 220 and tail 240. Body 230 can be formed of any number for suitable materials. For example, body 230 can be made either of either a permanent monofilament suture material, such as polypropylene, or of an absorbable material, such as polyglycolic acid. Body 230 has a first side or face 232 and an opposing second side or face 234. First face 232 is typically a smooth surface, while the second face 234 has surface features for facilitating the engagement and coupling of the suture element 200 to a driving member of device 100. For example, second face 234 can have a plurality of ribs or ridges 250 formed along at least a portion of its length.

In the illustrated embodiment, ribs 250 are formed perpendicular to the two opposing side edges 236 of the body 230. As described in detail below, the formation of ribs 250 permits suture element 200 to be driven in a ratcheting manner via a driving gear that is part of a ratchet like drive assembly of device 100. Ribs 250 can thus be thought of as being a gear rack defined by teeth. Accordingly, it is contemplated by the present disclosure for ribs 250, when present, to have any desired orientation and/or configuration sufficient to engage with and be driven by device 100.

In use with device 100 disclosed herein and during application to the surgical site, suture element can be formed into a resulting loop where first face 232 represents the outside surface of the resulting loop and second face 234 represents the inside of the resulting loop. Stated another way, suture element 200 can be applied to the tissue in a loop form so that second face 234 is proximate the incision being sutured, while first face 232 is remote from the incision. Of course, it is contemplated by the present disclosure for first face 232 to be proximate the incision or for either of edges 236 to be proximate the incision.

Tail 240 of suture element 200 has an opening 242 formed therein. Opening 242 includes a ratcheting lock, generally shown at 244, that is configured to engage and lock with the ribs 250 of body 230. Ratcheting lock 244 and ribs 250 operate so that once the needle 220 passes through opening 242 and past the ratchet 244, the needle is prevented from being pulled back through the opening due to the intimate meshing between the ribs 250 and the ratchet 244. Consequently, suture element 200 can be formed into the resulting loop, which may only be pulled tighter as a result of ribs 250 and ratcheting lock 244 being formed to only allow advancement of needle 220 and body 230 in one direction through opening 242.

Since suture element 200 assumes a locked position only if needle 220 advances through opening 242 and ribs 250 engage ratcheting lock 244, the registration of the needle with the opening is necessary to form the resulting loop and to allow the suture element to be tightened to the closed, locked position. In particular, if needle 220 is not properly aligned with opening 242, the needle 220 will not enter the opening correctly and instead, will either contact body 230 or will miss tail 240 and be loose. Advantageously, device 100 is configured to ensure the desired registration between needle 220 and opening 242.

Just as conventional sutures and needles come in many different sizes or gauges (i.e., 0, 2-0, 4-0, etc.), suture element 200 can be formed in many different sizes. Smaller sizes are more appropriate for fine tissue structures, while larger sizes provide greater strength and are more appropriate for heavier tissue structures.

In some embodiments, suture element can include a first locating feature 260, which assists device 100 to ensure proper registration between needle 220 and opening 242. First locating feature 260 can be defined at tail 240 and interacts with features of device 100 to ensure that the tail is maintained in the proper, desired position, while needle 220 is advanced toward through opening 242. In the illustrated embodiment, first locating feature 260 is in the form of a shoulder 262 that is defined between tail 240 and body 230. Unlike needle 220 and body 230, which have at least substantially equal thicknesses, tail 240, in this embodiment, has a greater thickness resulting in shoulder 262 being formed to provide a vertical wall 264 extending from first face 232 of the body. It will be appreciated that opening 242 extends through the increased thickness tail 240.

As described in detail below, first locating feature 260 engages a complementary second locating feature of apparatus 300 to cause tail portion 240, and in particular, opening 242 thereof, to be maintained in a known, desired location so as to allow needle 220 to be advanced by the apparatus into opening 242. In other words, tail portion 240 is held at a desired position where proper registration between opening 242 and needle 220 results to ensure the looping of suture element 200 and the interaction between ribs 250 and ratcheting lock 244.

It will be appreciated that suture element 200 and portions thereof can be formed of any number of different materials that are suitable for use with the device 100 and are suitable for use at the intended surgical site.

For example, at least the portions of suture element 200 that remain in the tissue, namely tail 240 and portions of body 230, can be fabricated of either bioabsorbable polymeric resins or of non-bioabsorbable biocompatible materials. Suitable bioabsorbable polymeric resins include, for example, homopolymers and copolymers derived from monomers selected from the group consisting of glycolic acid, glycolide, lactide, lactic acid, 1,4-dioxepan-2-one, p-dioxanone, ε-caprolactone, trimethylene carbonate and mixtures thereof.

Examples of suitable non-bioabsorbable biocompatible materials include homopolymers and copolymers of polypropylenes, silks, polyamides, polyesters, polyvinyl chlorides, polytetrafluoroethylenes, polysulfones, and mixtures thereof.

Suture element 200 can also include one or more suitable dyes, coatings, plasticizers, fillers, etc., as desired or appropriate to improve the visibility and/or handling characteristics of the element.

It will be understood that the above materials are merely exemplary in nature and any number of other materials can be used to manufacture suture elements 200 so long as the suture element is suitable for the intended application. Suture element 200 can be manufactured using any number of different conventional techniques, including an extrusion process or using a mold dies to form a solid molded structure.

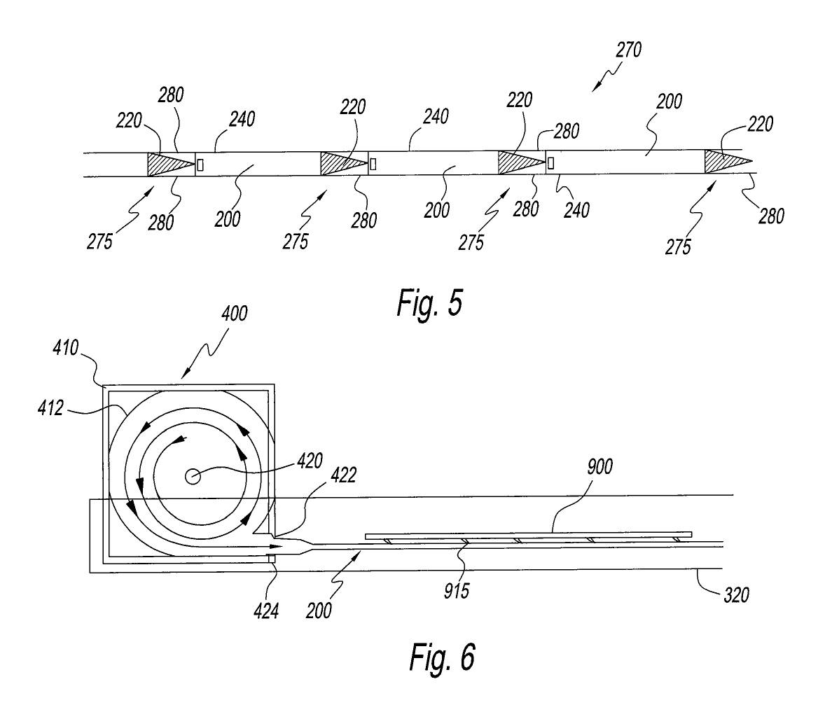

Referring now to FIG. 5, suture element 200 is shown connected to or arranged with a plurality of suture elements, which permit the elements to be easily loaded into device 100. More specifically, the individual suture elements 200 can be connected together to form an elongated interconnected supply 270 of elements. Within interconnected supply 270, suture elements 200 are connected together by a connection 280 in a tip-to-tail manner such that tail 240 of one suture element 200 is coupled to needle 220 of another suture element 200. Connection 280 is rupturable or breakable so as to allow the separation of one suture element 200 from interconnected supply 270.

In the illustrated embodiment, interconnected supply 270 includes one or more frangible or rupturable areas 275 connecting needle 220 to tail 240. For example, frangible areas 275 can include one filament 280 formed close to one edge 236 of suture element 200, while the other filament 280 can be formed close to the other edge 236 of the suture element. Filaments 280 can be formed of polymeric materials or other materials and can be formed of the same material that is used to manufacture suture element 200 itself or they can be formed of a different material.

It should be recognized that suture elements 200 are disclosed by way of example only being interconnected into interconnected supply 270 in a tip-to-tail manner. Of course, it is contemplated by the present disclosure for suture elements to be interconnected in any desired manner, such as a side-to-side manner where tails 240 and/or needles 220 of adjacent suture elements 200 are connected to one another.

Further, it should be recognized that suture elements 200 are disclosed by way of example only being interconnected into interconnected supply 270 by filaments 280. Of course, it is contemplated by the present disclosure for suture elements 200 to be coupled to one another by any suitable frangible area 275. For example, frangible area 275 can include weakened areas such as, but not limited to score lines or perforations, formed along the length of the interconnected suture elements 200. In this manner, frangible areas 275 can be formed strategically along the length of the interconnected suture elements 200 to permit individual suture elements 200 to be severed from the rest of the interconnected supply 270.

Advantageously, interconnected supply 270 of suture elements 200 can be loaded into any number of different types of structures, including different types of cartridges. For example, FIG. 6 illustrates interconnected supply 270 being coiled within a cartridge 400. Cartridge 400 includes a hollow housing 410 that can have any number of different shapes. In the illustrated embodiment, housing 410 has a square shape and since the interconnected suture elements 200 are in a coil configuration, inner edges 412 of the housing 410 are rounded to facilitate the unrolling of interconnected supply 270. Cartridge 400 can include a center spool 420 in housing 410 to facilitate the unrolling of the interconnected supply 270 similar to how a standard tape dispenser operates.

In an alternative configuration, interconnected supply 270 may be stored within the cartridge 400 in a linear, curved, or serpentine orientation to facilitate their travel into device 100.

Housing 410 includes a slot 422 that is formed therein to allow the interconnected supply 270 to exit the housing. In the illustrated embodiment, slot 422 is located in a bottom corner 424 of housing 410. Slot 422 can have a height sufficient to allow suture element 200 to freely pass therethrough and be fed through into device 100 as described below.

In some embodiments, interconnected supply 270 is maintained within housing 410 such that when suture elements 200 are discharged from the housing, ribs 250, formed along face 234 of body 230, are properly oriented and faces a drive mechanism of device 100, as described below, to permit advancement of the suture elements into the device.

Now that the construction and packaging of suture element 200 within cartridge 400 has been discussed, the construction and operation of applicator 300 shall be described with simultaneous reference to FIG. 1 and FIGS. 7 through 12.

Applicator 300 has a first or proximal end 302 and an opposing second or distal end 304. Applicator 300 includes an actuator handle 310, an at least partially hollow shaft or barrel 320 that extends outward from the handle, and a movable tip member 340 extending from the shaft.

Handle 310 is disposed at the first end 302 and includes a first part 312 that is a vertical part that houses a trigger lever 330 and a second part 314 that is a horizontal part that is attached to the first part 312. Optionally, handle 310 can have a secondary handle member 316 to facilitate grasping and holding of applicator 300. Secondary handle member 316 is formed perpendicular to one side of second part 314 and extends outward therefrom. In some embodiments, cartridge 400 can be also perform the function of secondary handle member 316.

In this manner, applicator 300 can be held in a pistol grip with one hand grasping handle member 310 and trigger lever 330. If needed, a second hand may be used to stabilize shaft 320 either directly on the shaft or, when present, on secondary handle member 316 or cartridge 400. Trigger lever 330 extends within first part 312 and can be depressed to cause actuation of a suture drive mechanism within apparatus 300 and movement of tip member 340.

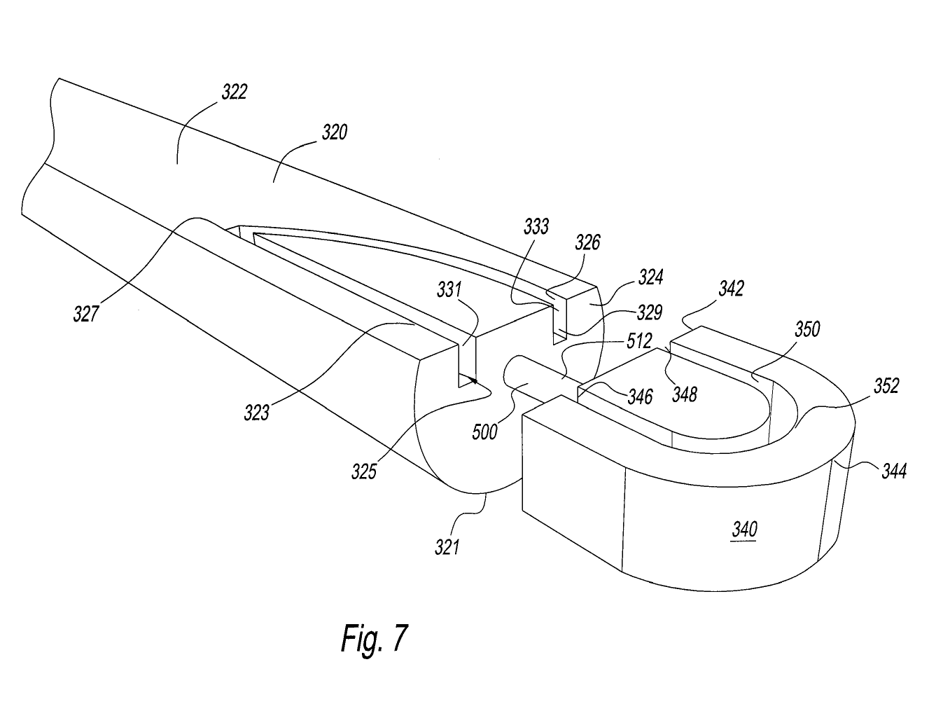

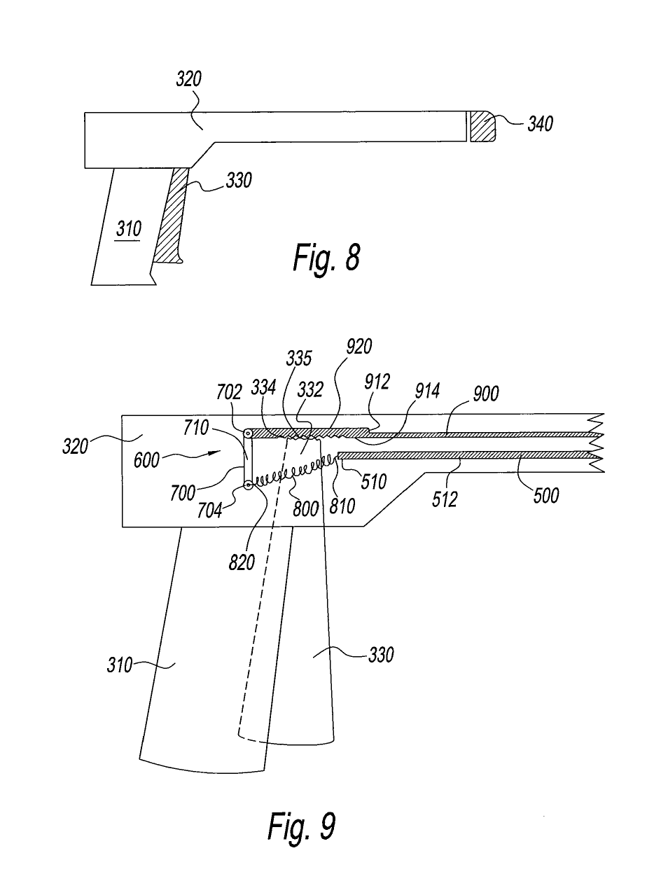

Shaft 320 extends outward from handle 310 and includes a first or proximal end 322 and an opposing second or distal end 324. First end 322 of shaft 320 interfaces with handle 310. At second end 324 of shaft 320, tip member 340 is provided and is moveable between an open, extended position, shown in FIGS. 1 and 7, and a closed, retracted position, shown in FIG. 8.

Tip member 340 assumes the open, extended position of FIG. 1 when apparatus 300 is in a ready state, prior to actuation of trigger lever 330. Shaft 320 can have any number of different shapes including the shape shown in FIG. 1, which illustrates a substantially planar top surface 322 and a curved or convex bottom surface 321.

Shaft 320 includes a main guide channel 325 in which interconnected supply 270 of suture elements 200 are advanced from cartridge 400 and a return channel 329 into which the suture element is delivered after passing through the second tissue segment to complete the suturing.

Tip member 340 is coupled to shaft 320 in such a manner that the tip member can be moved between the open, extended position and the closed, retracted position. In the illustrated embodiment, the movement of tip member 340 is linked to trigger lever 330.

Top surface 322 of shaft 320 is not entirely closed but rather, the top surface is at least partially open in the region of channel 325. In the illustrated embodiment, top surface 322 is slotted and in particular, top surface 322 includes a first slot 323 and a second slot 326 that intersects the first slot at an intersection point 327 that is spaced from second end 324 of shaft 320. However, second slot 326 is spaced from or does not intersect with first slot 323 at second end 324 so as to form open ends 331 and 333.

As best shown in FIG. 7, first slot 323 overlies main guide channel 325 and second slot 326 overlies return channel 329, with first and second slots 323, 326 being open at second end 324 of shaft 320. As described herein, channels 325, 329 serve as guide channels for guiding suture element 200 in a controlled manner.

First slot 323 is axially aligned with and overlies guide channel 325 and therefore, interconnected supply 270 is driven and advanced within the guide channel 325 so that a leading suture element 200 on the supply is accessible through the first slot 323.

Tip member 340 is operatively coupled to the shaft 320 by a first drive element 500 that causes the controlled movement of the tip member from the open, extended position of FIGS. 1 and 7 to the closed, retracted position of FIG. 8.

First drive element 500 is operatively connected to trigger lever 330 so that when the trigger lever is actuated, the first drive element is driven within shaft 320. In the illustrated embodiment, first drive element 500 is in the form of a pull rod 512 that extends within the interior of shaft 320 and extends through an opening at the second end 324 of the shaft 320. Pull rod 512 moves linearly within the interior of shaft 320 and as shown in FIG. 7, the pull rod is disposed between the channels 325, 329 formed in the shaft 320.

Tip member 340 is complementary to shaft 320 and has a first or proximal end 342 and an opposing second or distal end 344. Proximal end 342 complements the second end 324 of shaft 320 and in the illustrated embodiment, both the proximal end 342 and the second end 344 are planar ends. Tip member 340 has a curved shape so that distal end 344 has an arcuate or semi-circular shape. In addition, tip member 340 can have a cross-section that is same or different than the cross-section of shaft 320.

Tip member 340 has complementary channel and slot structures relative to the channel and slot structures of the shaft 320. More specifically, tip member 340 includes a u-shaped channel 350 with two open ends 346, 348 being formed at the proximal end 342 of the tip member 340. Channel 350 is not open at distal end 344 but instead is defined by a closed arcuate slot. Between open ends 346, 348 of channel 350, tip member 340 has a solid structure with pull rod 512 being attached to the tip member between the open ends of the channel. Tip member 340 further includes a third slot 352 that overlies channel 350 so that suture element 200 is accessible through the third slot 352.

Channel 350 serves as a guide or load channel for suture element 200 with the open ends 346, 348 thereof being axially aligned with open ends 331, 333 of channels 325, 329. In other words, if proximal end 342 of the member 340 is placed adjacent distal end 344, open ends 331, 333 of channels 325, 329 align with open ends 346, 348 of channel 350. In this manner, suture element 200 can be advanced through the suture channel 325, around return channel 350, and ultimately into return channel 329.

In the open, extended position shown in FIGS. 1 and 7, proximal end 342 of tip member 340 is spaced a first prescribed distance from second end 324 of shaft 320. The first prescribed distance is of a sufficient distance that segments of incised tissue can be received therein. In one embodiment, proximal end 342 of tip member 340 is spaced about 1.0 centimeter (cm) away from second end 324 of shaft 320. In contrast, in the closed or retracted position shown in FIG. 8, proximal end 342 of tip member 340 is spaced a second prescribed distance from second end 324 of shaft 320. The second prescribed distance is of a sufficient distance that segments of incised tissue can be retained between tip member 340 and shaft 320.

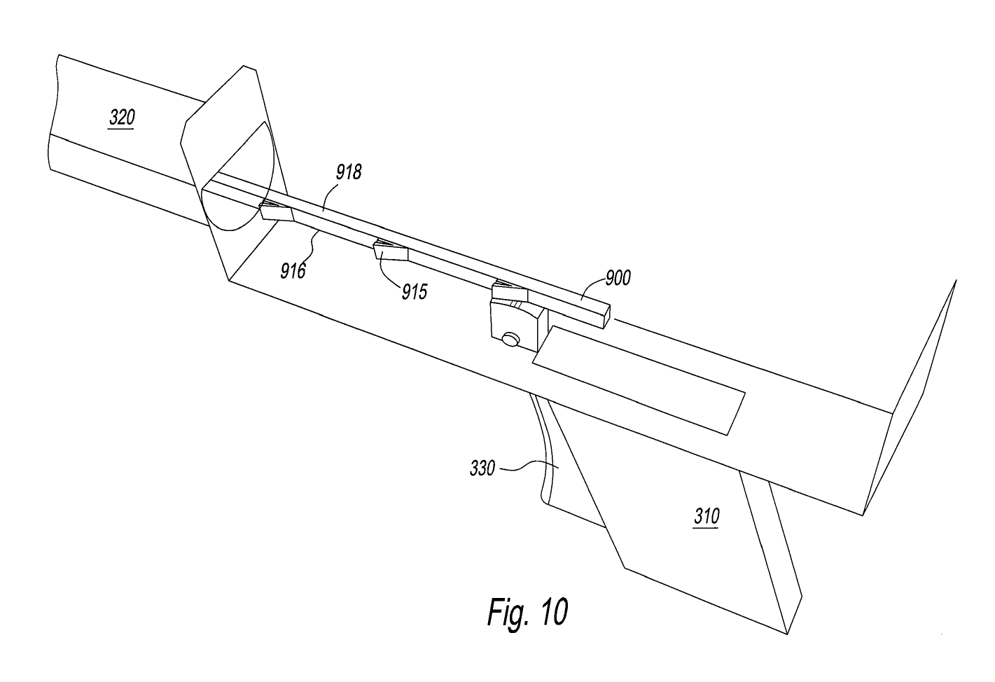

FIGS. 8 and 9 show a drive mechanism 600 for actuating apparatus 300 and moving tip member 340 between the open, extended position and the closed, retracted position. More specifically, trigger lever 330 can be squeezed and is preferably biased within handle 310 so that trigger lever 330 returns to a normally extended position and is ready to be squeezed again for further activation of apparatus. In the ready state, prior to actuation of trigger lever 330 with tip member 340 in the open, extended position, trigger lever 330 is fully extended. Trigger lever 330 can be pivotally coupled to the handle 310 at a pivot 332 that is near a first end 334 of the trigger lever. In addition and according to one embodiment, the first end 334 of the trigger lever 330 is slightly curved and includes a pinion gear 335.

Drive mechanism 600 also includes a pivotable link 700 that has a first end 702 and an opposing second end 704. Pivotable link 700 is coupled to the handle 310 at a pivot 710 that is centrally located along the length of the link. In a rest position, link 700 is generally vertically oriented within the handle 310. In the illustrated embodiment, link 700 is disposed behind the first end 334 of trigger lever 330.

First drive element 500, in the form of pull rod 512, has a first end 510 that is operatively coupled to link 700 and a second end 520 that is coupled to tip member 340 as discussed above. More particularly, first end 510 is connected to a first end 810 of a biasing member 800 and an opposite second end 820 of the biasing member is attached to second end 704 of link 700 such that when the link pivots about pivot 710, biasing member 800 is moved from a normal, unbiased state to a biased or energized state. In the illustrated embodiment, biasing member 800 is in the form of a coil spring.

Drive mechanism 600 also includes a second drive element 900 that, like first drive element 500, is in the form of an elongated push rod 902 having a first end 910 and an opposing second end 904. Second drive element 900 also has a first or top face 912 and an opposing second or bottom face 914. First end 910 is coupled to first end 702 of link 700. Second drive element 900 has a third or side face 916 and a fourth or side face 918.

As described below, second drive element 900 is configured to controllably advance suture element 200 within apparatus 300. At least part of drive mechanism 600 for driving suture elements 200 is in the form of a rack and pinion arrangement. As is known, a rack and pinion is a pair of gears which convert rotational motion into linear motion. Circular pinion 335 engages teeth 920 that are located along second face 914 of second drive element 900 proximate first end 910 thereof. Second drive element 900 thus functions as the rack and rotational motion applied to pinion 335 will cause second drive element 900 to move linearly, up to a limit of its travel.

Teeth 920 are constructed to mesh with the pinion 335 so that rotation of the pinion causes the pinion teeth to mesh with teeth 920 and drive second drive element 900 linearly.

As shown, the thickness of second drive element 900 can be non-uniform along its length. In particular, the portion of the second drive element 900 that includes the teeth 920, including first end 910 thereof, can have a greater thickness compared to other portions of second drive element 900. However, it will be understood that second drive element 900 can have a uniform thickness, equally as well.

Third face 916 of second drive element 900 includes ratchets 915 that are spaced apart and are formed in only one direction. One-way ratchets 915 engage ribs 250 that are formed on the suture element 200 in the forward direction only when trigger lever 330 is squeezed. Upon release of trigger lever 330, one-way ratchets 915 are configured to slide back over suture element 200 to allow further engagement of second drive element 900 with suture element 200. FIG. 10 shows this arrangement; however, it will be understood that FIG. 10 does not show suture element 200 for reasons of clarity.

Interconnected supply 270 is drawn from cartridge 400 and is fed into a feed channel (not shown) that is located adjacent second drive element 900. Teeth 250 of suture elements 200 in interconnected supply face one-way ratchets 915 of second drive element 900 to allow engagement therebetween and driving of the suture elements 200 within shaft 320.

Both the pull rod 512 and the push rod 902 can be located within a guide channel or retaining structure (not show) that maintain the pull rod 512 and push rod 902 in the desired horizontal orientation within shaft 320 and ensures a smooth operation. As shown in FIG. 9, pull rod 512 and push rod 902 are disposed at least substantially parallel to one another, with the push rod being located above the pull rod.

As push rod 902 is driven linearly, suture element 200 is likewise driven linearly within apparatus 300 and in particular, within shaft 320. The interaction between push rod 902 and suture element 200 results in continued advancement of the suture element within shaft 230 and into channels 325, 350, and 329.

When trigger lever 330 is squeezed or actuated, link 700 pivots in a clockwise direction about pivot 710 and resulting in biasing member 800 being extended such that energy is stored by the biasing member. Since first end 810 of biasing member 800 is coupled to pull rod 512, the extension of the biasing member causes retraction of the pull rod 512.

Pull rod 512 represents the tip retracting member of drive mechanism 600 since linear motion of the pull rod is translated into the retraction and extension of tip member 240. More particularly, when trigger lever 330 is squeezed and link 700 rotates in a clockwise direction, pull rod 512 is moved from the extended or rest position of FIGS. 1 and 7 to the retracted position of FIG. 8. At the same time, the squeezing of trigger lever 330 causes the advancement of push rod 902 within shaft 320. In other words, the squeezing of trigger lever 330 results the simultaneous movement of both push rod 902 and pull rod 512 in opposite directions.

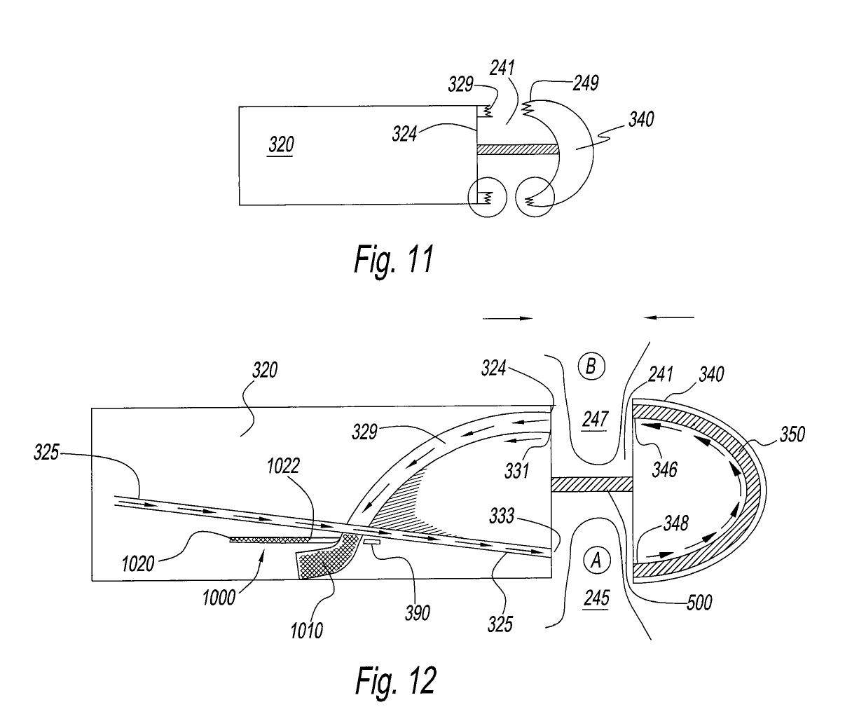

FIG. 11 illustrates the tip member 340 in the open, extended position with a gap 241 being formed between open tip member 340 and second end 324 of shaft 320. As mentioned above, gap 241 is of sufficient size to receive tissue to be sutured. More specifically, tissue to be sutured includes a first tissue segment 245 and a second tissue segment 247. First tissue segment 245 is inserted in gap 241 on one side of pull rod 512 and second tissue segment 247 is inserted in gap 241 on the other side of the pull rod. As the tip member 240 is moved to the closed, retracted position of FIG. 8 due to the retraction of pull rod 512, tissue segments 245, 247 are pinched between tip member 340 and second end 324 of shaft 320.

The clamping force of apparatus 300 between tip member 340 and second end 324 is provided by the biasing effects of biasing member 800, which mitigates pinching or crushing damage to tissue segments 245, 247. In some embodiments, the biasing force of biasing member 800 and/or gap 241 between tip member 340 and second end 324 can be adjusted by the user.

Cartridge 400 can be loaded into apparatus in any desired manner. For example, cartridge 400 is inserted into a slot or the like formed in handle 310 and as shown in FIG. 1, the cartridge can be inserted into a side of the handle such that it is perpendicular thereto. In this manner, cartridge 400 is easily accessible and can be easily inserted and removed from handle 310 as needed. Suture elements 200 unroll from cartridge 400 into shaft 320 due to the engagement with push rod 902 of drive mechanism 600.

It will be understood that after first inserting or loading cartridge 400 into applicator 300, trigger lever 330 will likely need to be squeezed multiple times until the first suture element 200 of interconnected supply 270 is near open end 333 of shaft 320. Once the leading suture element 200 assumes this position, device 100 can be introduced into the surgical field where it is to be used. Alternatively, the initial loading step can be performed at the surgical field by an assistant or the like. Applicator 300 can then be placed directly into the wound, as in open surgery, or inserted through a laparoscopic port to be used for laparoscopic surgery.

FIG. 12 shows the path of interconnected supply 270 of suture elements 200 within shaft 320 and tip member 340. Channel 325 for interconnected supply 270 of suture elements 200 is located along the center of the instrument and then angles toward one edge of the instrument until the suture element exits open end 333 of shaft 320. Channel 325 and open end 333 direct suture element 200 to and through first tissue segment 245. After exiting first tissue segment 245, needle 220 of suture element 200 is received by open end 348 of movable tip 340, guided around channel 350 and out of open end 346 of the movable tip. Channel 350 and open end 348 direct suture element 200 to and through second tissue segment 247. After exiting second tissue segment 247, needle 220 of suture element 200 is received by open end 346 of channel 329. Next, needle 220 of suture element 200 is advanced through channel 329 until the needle reaches intersection point 327 where channel 329 crosses over and communicates with channel 325.

In one exemplary suturing process using device 100, applicator 300 is oriented so that first tissue segment 245 is contained within space 241. Trigger lever 330 is squeezed a first time to retract tip member 340, thereby compressing first tissue segment 245 between shaft 320 and tip member 340, while push rod 902 drives interconnected supply 270 along guide channel 325 within shaft 320 until needle 220 penetrates first tissue segment 245 and advances into guide channel 350 formed in the tip member 340. The trigger lever 330 is then released, thereby opening up the gap 241 between shaft 320 and tip member 340. Here, first tissue segment 245 is held in place since suture element 200 has penetrated therethrough much like a conventional suture that has already been manually passed through one piece of the tissue.

Next, second tissue segment 247 is brought into gap 241 at an opposite end and trigger lever 330 is squeezed a second time to cause a retraction of tip member 340 and compression of second tissue segment 247 between shaft 320 and tip member 340. Push rod 902 again advances interconnected supply 270 so that suture element 200 is driven along shaped guide channel 350 until needle 220 penetrates second tissue segment 247 and advances into channel 329 formed in shaft 320. Again, trigger lever 330 is then released, thereby opening up the gap 241 between shaft 320 and tip member 340. Here, first and second tissue segments 245, 247 are held in place since suture element 200 has penetrated therethrough much like a conventional suture that has already been manually passed through both pieces of tissue.

Finally, trigger lever 330 is squeezed a third time to cause push rod 902 to advances interconnected supply 270 so that suture element 200 is driven along channel 329 until needle 220 is fed through opening 242 of tail 240 and at least one of ribs 250 is engaged by ratcheting lock 244. In this position, suture element 200 is locked in a looped position through first and second tissue segments 245, 247. However, suture element 200 has not yet been tightened. Rather, and advantageously, device 100 allows multiple suture elements 200 to be inserted before being tightened to close the incision in the tissue.

Apparatus 300 also, in some embodiments advantageously, includes a needle disposal system 1000 that is configured to remove or separate needle 220 from body 230 after the needle has been passed through opening 242 of tail 240. Needle disposal system 1000 includes a collection receptacle or space 1010 and a cutting element 1020.

Collection receptacle 1010 is in communication with the end of return channel 329 and slot 326 so that once needle 220 is separated from 230, the needle is deposited into the receptacle for collection thereof. Collection receptacle 1010 thus stores the used needles 220 of suture elements 200 that have been applied to the tissue segments of the incised tissue. Advantageously, removal of the sharp needle 220 from suture element 200 after formation and locking of the loop but before removal of the loop from apparatus 300 mitigates exposure of the surgical staff to needle stick injuries.

Cutting element 1020 has a sharp end 1022 that is constructed to cut through suture element 200 at a location that is at or near the interface between the needle 220 and body 230. Cutting element 1020 can be guided to ensure proper linear movement and an anvil or back plate can be provided so that needle 220 is between the back plate and retracted cutting element 1020, which is then advanced and driven into needle 220 to sever it from body 230 of suture element 200 with the back plate providing support. In one embodiment, second locating feature 390 can serve as a back plate.

Cutting element 1020 can have a metal tip or be of some other type of material that is sharp and strong enough to sever needle 220 from body 230. Alternately, cutting element 1020 can use energy such as but not limited to heat energy, vibratory energy, ultrasonic energy, light energy and any combinations thereof, to sever needle 220 from body 230.

It will be appreciated that cutting element 1020 can be manually operated by the operator by using a slide button of the like that located along the side of the shaft. In this manner, once needle 220 has passed through opening 244 and at least one rib 250 has engaged the locking ratchet 244, the operator can activate the cutting element 1020 to cause severing of needle 220.

Alternatively, cutting element 1020 can be part of the automatic process and be operatively linked to drive mechanism 600 that advances the suture elements. In this manner, the third actuation of trigger lever 330 that inserts needle 220 through opening 242 can also cause the activation of cutting element 1020. Here, cutting element 1020 can be biased so that once the cutting operation is performed, the cutting element 1020 returns to a retracted position where it is ready to be driven again into an intact suture element 200 for severing needle 220 therefrom.

After needle 220 is severed from suture element 200, the remaining portions of the suture element 200, namely body 230 and tail 240 remain contained within channels 325, 350, 329. Since channels 325, 350, 329 are open along the top of apparatus 300 via slots 323, 326, 352, the looped and locked, but not tightened suture element 200 can easily be removed from apparatus 300 by moving the apparatus so that the suture element exits through the open top of shaft 320 and movable tip 340. Here, the action of withdrawing apparatus 300 from the looped and locked, but not tightened suture element 200 is sufficient to break or sever connection 280 of interconnected supply 270 so as to separate the looped suture element 200 from the interconnected supply. Of course, it is contemplated by the present disclosure for connection 280 of interconnected supply 270 to be severed by any desired mechanism.

After ejection of the looped suture element 200, the next-in-line suture element 200 of interconnected supply 270 is ready for advancement into the open, extended tip member 340 in the manner described above.

Again, suture element 200, which has been applied to the surgical site, remains in a looped and locked position with one or more ribs 250 of body 230 being intimately engaged with locking ratchet 244 in opening 242 of tail 240. The looped suture element 200 can either be tightened immediately after being applied to the tissue incision or all of the suture elements 200 can be applied along the incision and then each is individually tightened in succession to complete the suturing operation. In some instances, it may be required to trim excess portions of body 230 from suture element 200 after the suture element has been fully tightened.

It should be recognized that the operation of device 100 has been described above by way of example only as requiring three separate activations of trigger lever 330. However, this exemplary embodiment has merely been provided as a way for device 100 to mimic the suturing steps of current manual interrupted suturing processes. Of course, it is contemplated by the present disclosure for device 100 to have any desired structure sufficient to advance interconnected supply 270 from cartridge 400 through channels 325, 350, and 329 until needle 220 is looped through opening 242 of tail 240 and at least one of ribs 250 is engaged by ratcheting lock 244.

For example, while a linear ratchet type advancement mechanism has been described herein to advance interconnected supply 270, it will be understood and appreciated that other types of mechanisms can be used to advance the suture elements in a controlled manner. For example, instead of using a manual gear based mechanism as disclosed herein, interconnected supply 270 can be advanced in an automatic manner as by using a motor-driven mechanism involving one or more gears, the teeth of which engage with the ribs or ridges 250 on suture element 200. Such a motor could be activated through a switch in the handpiece of the device in lieu of or in addition to mechanical force provided by handle 330. Such gear-driven or motorized force can be used as the exclusive propulsion force advancing interconnected supply 270 or can be used in conjunction with direct manually-applied force through the ratchet mechanism described herein. The motor and gears can be included within the main body of the apparatus 300 and/or within tip member 340.

Thus, it will be appreciated that the drive mechanism can either be either a purely manual mechanism, much like the ratcheting operation of a standard caulking gun; or can be an automatic mechanism; or a combination thereof. When an automatic mechanism is employed, a motor and associated controller (e.g., an electronic circuit) are used to drive interconnected supply 270. For example, a stepper motor or the like or other type of motor can be used to controllably advance the leading suture elements a precisely defined distance that properly advances the leading suture element through the tissue segments, the tip member, and into a loaded position with respect to the cutting element.

It will further be appreciated that a drive mechanism can be employed in which the leading suture element is severed from the other interconnected suture elements prior to being driven into the first tissue segment and subsequently loaded into the tip member. For example, another cutting element or the like can be used to sever the leading suture element from the interconnected suture elements and the severed leading suture element is then loaded and driven along a load channel toward the tip member and into contact with the tissue segments in the manner described above. In this embodiment, any number of different drive mechanisms can be employed to ensure that the severed suture element is driven toward and within the guide channel of the tip member, thereby suturing the tissue in the manner described herein.

In some embodiments, shaft 320 includes a second locating feature 390, which is configured to interact with first locating feature 260 of suture element 200. Second locating feature 390 can be part of and integral with shaft 320, which creates an interference with first locating feature 260 to limit the travel of the leading suture element 200 of interconnected supply 270 within main guide channel 325. The length of suture element 200, the location of first locating feature 260, and the location of second locating feature 390 are configured so that when first and second locating features 260, 390 contact one another, there is sufficient length of the suture element 200 that needle 220 can be driven and pass through opening 242 of tail 240 so as to form the loop in suture element 200 with at least one of ribs 250 engaged by ratcheting lock 244.

In other words, the forward advancement of suture element 200 is limited by interaction first and second locating features 260, 390, which ensures that opening 244 of tail 240 is located at a position where the opening is axially aligned and in communication with one end of channel 329 to allow free passage of needle 220 into and through the opening.

In terms of the placement of suture elements 200, approximately the same number of suture elements 200 is used in the suturing process of the present disclosure compared to a traditional manual suturing technique. For example, a suture element 200 can be applied every 1 to 2 centimeters along the surgical site (e.g., an incision).

During use of device 100, the tissue segments are firmly held by the device and in particular, the tissue is firmly held between the closed, retracted tip member 340 and shaft 320. In one embodiment, apparatus 300 can include a first gripping member 249 on the proximal side of tip member 340, which engages a second gripping member 329 of second end 324 of shaft 320. First and second gripping members 249, 329 can be in the form of teeth that are constructed so that they do not damage the tissue when tip member 340 is in the closed, retracted position. First and second gripping members 249, 329 can be made of a hard material, such as plastic or metal, or can be formed of a softer material, such as rubber. Typically, one side of the gripping member (e.g., right side or left side) are used to grasp tissue at a time, although the left and right gripping member can also be used to simultaneously grasp the tissue.

It will be appreciated that the tip member 340 can include a member to facilitate the movement of the suture element 200 along the guide channel 350. For example, a gear or the like (not shown) can be included within the tip member 340 in communication with the guide channel 350 so that the teeth of the gear mesh with the teeth formed along the body 230 of the suture element 200. This gear can be driven either directly or indirectly by the mechanism 600 that is contained in the device 100. The driving of the gear causes forward advancement of the suture element 200 through the guide channel 350. Alternatively, a smooth roller or other curved advancing mechanism may be placed in the tip member 340 to facilitate return of the body 230 of the suture element 200.

The guide channel 350 can also be formed so that it has smooth, polished surfaces which reduce the friction between the suture element 200 and the guide channel 350 as the suture element 200 is driven therein.

It will also be appreciated that the present device is not limited to having the disclosed tip member 340 but instead, other types of tip members can be provided so long as they perform the intended function described herein and guide the suture element after it has passed through the first tissue segment so that it is looped back through the second tissue segment. For example, the tip member can include an opposing pair of tissue receiving slots that receive the first and second tissue segments. Instead of having the entire tip member being extendable and retractable, the tip member can include discrete movable gripper members (e.g., gripping fingers or posts) that are configured to grip the tissue segments at localized areas or points after insertion of the tissue within the respective slot. The gripper members can be activated manually or in an automatic manner.

Applicator 300 can be formed of any number of different materials so long as they are suitable for use in the intended application. For example, applicator 300 can be formed of a suitable plastic material and due to the sanitary considerations and applicable regulations, the applicator 300 can be manufactured as a disposable item in that after suture elements 200 are applied, the applicator is discarded. More specifically, cartridge 400 can contain a suitable number of suture elements 200 for a standard suturing operation and therefore, both cartridge 400 and applicator 300 can be discarded after the suturing operation is performed. In one embodiment, cartridge 400 can contain fifteen (15) suture elements.

Alternatively, device 100 may be fabricated from reusable materials including metal and plastic that may be sterilized for repeated usage. This would allow the device to be partially or wholly reusable or “reposable” and would decrease the amount of waste material produced with each use.

It will be appreciated that device 100 can be provided in a sterile foil package to permit device 100 to be opened on the surgical field using conventional sterile techniques. Suture elements 200, prepackaged in cartridge 400 can be included in the same sterile foil package or can be packaged separately (e.g., another sterile foil package) and can be loaded into the device 100 separately at the surgical field.

In some embodiments of the present disclosure, at least one cartridge 400 with interconnected supply 270 therein can be provided in a first sterile kit. In other embodiments of the present disclosure, apparatus 300 can be provided in a second sterile kit. In yet another embodiments, apparatus 300 and at least one cartridge 400 with interconnected supply 270 can be provided in a third sterile kit.

Device 100 can be used in open surgery where it will provide the benefit of increased speed of tissue apposition and decreased exposure of needles to the patient, surgeon, and surgical team. Device 100 also finds utility in laparoscopic surgery, where knot tying and suture manipulation are particularly difficult. In fact, suturing and tying are the most complex and time-intensive tasks performed in advanced laparoscopic procedures. Device 100 is also useful in open surgery in anatomic areas where conventional knot-tying can be difficult such as at the bottom of a narrow, deep surgical field.

It should also be noted that the terms “first”, “second”, “third”, “upper”, “lower”, and the like may be used herein to modify various elements. These modifiers do not imply a spatial, sequential, or hierarchical order to the modified elements unless specifically stated.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. An interrupted suturing device, comprising:
a cartridge having a plurality of elongated suture elements interconnected to form an interconnected supply of suture elements, each suture element having a needle at one end, a ratchet opening at an opposite end, and a body between the needle and the ratchet opening; and
an applicator that receives the cartridge and includes a drive mechanism for driving the interconnected supply along a shaft of the applicator to a movable tip thereof, the movable tip being operatively coupled to the drive mechanism such that the movable tip moves between an open, position and a closed position relative to the shaft, wherein the shaft includes a first channel architecture and the movable tip includes a complementary second channel architecture that allows a lead suture element of the interconnected supply to pass through a first tissue segment disposed within a space between the shaft and movable tip before being passed through a second tissue segment disposed within the space after traveling within the second channel architecture, the first channel architecture including a channel segment that directs the needle of the lead suture element to pass through the ratchet opening formed at the tail thereof so as to create a looped suture element.

2. The interrupted suturing device of claim 1, further comprising a needle disposal system configured to remove or separate the needle from the body after the needle has been passed through the ratchet opening of the tail.

3. The interrupted suturing device of claim 2, wherein the needle disposal system comprises a needle collection receptacle and a needle cutting element.

4. An interrupted suturing apparatus for applying suture elements to tissue, comprising:
an actuator handle;
an at least partially hollow shaft that extends outward from the actuator handle;
a tip connected to the hollow shaft;
a drive mechanism for driving an interconnected supply of the suture elements through the shaft to the tip, the actuator handle being configured to activate the drive mechanism;
a plurality of open topped channels defined in the shaft and the tip that directs the interconnected supply to form a looped suture element from a first suture element of the interconnected supply;
a needle cutting element in the hollow shaft, the needle cutting element being configured to cut a portion of the first suture element from remaining portions of the first suture element; and
a needle collection receptacle in the hollow shaft, the needle collection receptacle being configured to receive the portion of the first suture element.

5. The interrupted suturing apparatus of claim 4, wherein the actuator handle is configured to activate the needle cutting element.

6. The interrupted suturing apparatus of claim 5, wherein the tip is movably connected to the hollow shaft between an open position and a closed position.

7. The interrupted suturing apparatus of claim 6, wherein the drive mechanism is operatively coupled to the tip such that the movable tip moves between the open and closed positions upon activation of the drive mechanism via the actuator handle.

8. The interrupted suturing apparatus of claim 7, further comprising a trigger lever in the actuator handle wherein the drive mechanism.

9. An interrupted suturing device for applying a suture element to tissue, comprising:
a handle that includes an actuator;
a shaft extending from the handle, the shaft including a suture channel that is open at two locations along a distal end of the shaft,
a tip member that is coupled to the shaft and movable between an extended position where the tip member is spaced from the distal end of the shaft and a retracted position, wherein a space is formed between the tip member and the distal end of the shaft in the extended position, the tip member having a return channel formed therein, the return channel being open at two locations along a proximal end of the tip member, the open ends of the suture channel being axially aligned with the open ends of the return channel; and
a drive mechanism operatively coupled to the actuator for driving the suture element along the suture channel and return channel, the tip member being operatively coupled to the drive mechanism to cause movement of the tip member between the extended position and the retracted position.

10. A cartridge for use with an interrupted suturing apparatus, comprising:
a plurality of elongated suture elements each having a needle at a head thereof, a ratchet opening at a tail thereof, and a body between the needle and the ratchet opening, the plurality of elongated suture elements being interconnected to define an interconnected supply of suture elements, wherein the ratchet opening is sized to receive the needle and the body therein; and
a housing having a hollow interior that contains the interconnected supply, the housing having a slot through which the interconnected supply exit.

11. The cartridge of claim 10, further comprising a frangible connection connecting the tail of a leading suture element to the needle of a following suture element.

12. The cartridge of claim 10, wherein the interconnected supply is coiled within the housing.

13. The cartridge of claim 10, wherein the needle is flexible about a first plane but is inflexible about a second plane.

14. The cartridge of claim 10, wherein the needle, the body, and the tail are formed of the same material.

15. The cartridge of claim 10, wherein the needle is made of a first material, while the body and the tail are formed of a second, different material.

16. The cartridge of claim 15, wherein the second material comprises polypropylene or polyglycolic acid and the first material comprises metal.

17. The cartridge of claim 10, wherein the needle is formed of a material that is radio-opaque.

18. The cartridge of claim 10, wherein at least the body and the tail are formed of bioabsorbable polymeric resins selected from the group consisting of homopolymers and copolymers derived from monomers selected from the group consisting of glycolic acid, glycolide, lactide, lactic acid, 1,4-dioxepan-2-one, p-dioxanone, ε-caprolactone, trimethylene carbonate and mixtures thereof.

19. The cartridge of claim 10, wherein at least the body and the tail are formed of non-bioabsorbable biocompatible materials selected from the group consisting of homopolymers and copolymers of polypropylenes, silks, polyamides, polyesters, polyvinyl chlorides, polytetrafluoroethylenes, polysulfones, and mixtures thereof.

20. The cartridge of claim 10, wherein the plurality of elongated suture elements comprise a visibility enhancing component selected from the group consisting of a dye, a coating, a plasticizer, a filler, and any combinations thereof.

21. The cartridge of claim 10, further comprising a plurality of ribs defined on the body, the ribs and ratchet opening being configured so that the body is slideable through the ratchet opening in only one direction.

22. A suture element comprising:
a needle having a tip that is sharp enough to facilitate passage through tissue;
a tail having ratchet opening; and
a body connecting the needle and the tail to one another, the body having a plurality of ribs defined thereon,
the needle being separable from the body, and
the plurality of ribs and the ratchet opening being configured so that the body is slideable through the ratchet opening in only one direction providing the suture element in a form of a knotless tissue apposition member.

23. The suture element of claim 22, wherein the needle is flexible about a first plane but is inflexible about a second plane.

24. The suture element of claim 22, wherein the needle, the body, and the tail are formed of the same material.

25. The suture element of claim 22, wherein the needle is made of a first material, while the body and the tail are formed of a second, different material.

26. The suture element of claim 25, wherein the second material comprises polypropylene or polyglycolic acid and the first material comprises metal.

27. The suture element of claim 22, wherein the needle is formed of a material that is radio-opaque.

28. The suture element of claim 22, wherein at least the body and the tail are formed of bioabsorbable polymeric resins selected from the group consisting of homopolymers and copolymers derived from monomers selected from the group consisting of glycolic acid, glycolide, lactide, lactic acid, 1,4-dioxepan-2-one, p-dioxanone, ε-caprolactone, trimethylene carbonate and mixtures thereof.

29. The suture element of claim 22, wherein at least the body and the tail are formed of non-bioabsorbable biocompatible materials selected from the group consisting of homopolymers and copolymers of polypropylenes, silks, polyamides, polyesters, polyvinyl chlorides, polytetrafluoroethylenes, polysulfones, and mixtures thereof.

30. The suture element of claim 22, wherein at least one of the needle, the body, and the tail comprise a visibility enhancing component selected from the group consisting of a dye, a coating, a plasticizer, a filler, and any combinations thereof.
